(19) United States
(12) Patent Application Publication
(10) Pub. No.: US 2020/0054752 A1
(43) Pub. Date: Feb. 20, 2020

(54) TITANIUM NITRIDE PLASMONIC NANOPARTICLES FOR CLINICAL THERAPEUTIC APPLICATIONS

(71) Applicant: Purdue Research Foundation, West Lafayette, IN (US)

(72) Inventors: Urcan Guler, Lafayette, IN (US); Alexander Kildishev, West Lafayette, IN (US); Gururaj Naik, West Lafayette, IN (US); Alexandra Boltasseva, West Lafayette, IN (US); Vladimir M. Shalaev, (US)

(73) Assignee: Purdue Research Foundation, West Lafayette, IN (US)

(21) Appl. No.: 16/665,319

(22) Filed: Oct. 28, 2019

Related U.S. Application Data
(63) Continuation of application No. 14/896,493, filed on Dec. 7, 2015.
(60) Provisional application No. 61/934,758, filed on Feb. 1, 2014.
(60) Provisional application No. 61/883,764, filed on Sep. 27, 2013.
(60) Provisional application No. 61/831,218, filed on Jun. 5, 2013.

(51) Int. Cl. A61K 41/00; A61N 5/06; A61K 9/00; A61K 9/51; A61K 47/69

(52) U.S. Cl. CPC A61K 41/0052; A61N 5/0625; A61K 9/0019; A61K 9/5115; A61N 2005/0643; A61K 47/6923; A61K 47/6905; A61K 9/0009; A61N 5/062; A61K 47/6929

(57) ABSTRACT

Disclosed herein are nanoparticle-based plasmonic solutions to therapeutic applications employing titanium nitride (TiN) and other non-stoichiometric compounds as the plasmonic material. Current solutions are suboptimal because they require complex shapes, large particle sizes, and a narrow range of sizes, in order to achieve plasmonic resonances in the biological window. The nanoparticles discloses herein provide plasmonic resonances occurring in the biological window even with small sizes, simple shapes, and better size dispersion restrictions. Local heating efficiencies of such nanoparticles outperform currently used Au and transition metal nanoparticles. The use of smaller particles with simpler shapes and better heating efficiencies allows better diffusion properties into tumor regions, larger penetration depth of light into the biological tissue, and the ability to use excitation light of less power.

### CROSS REFERENCE TO RELATED APPLICATIONS

The present application claims priority to, and incorporates fully by reference, U.S. Provisional Patent Application No. 61/831,218, filed Jun. 5, 2013, U.S. Provisional Patent Application No. 61/883,764, filed Sep. 27, 2013, and U.S. Provisional Patent Application No. 61/934/758, filed Feb. 1, 2014.

### FIELD OF THE INVENTION

The claimed invention relates to the field of plasmonics technology, impacting particular areas including, but not limited to, light-induced clinical therapeutic applications via the hyper-thermic effects of plasmonic nanoparticles and biological sensing applications via the near field enhancement effects of plasmonic nanoparticles.

### BACKGROUND OF THE INVENTION

Plasmonics technology relies upon the coupling of light into free electron plasma in metals to create a wave of surface charge oscillation called plasmon. Plasmon is typically associated with a highly concentrated electromagnetic field, which is a key feature in many of its applications.

Plasmon may exist only at or on the surface of a metal and is often referred to as surface plasmon. Thus, metal is an essential component of any plasmonic device. The optical properties of the metal used in a given plasmonic device will dictate the performance of the device. And since metals are often characterized by huge optical losses, this limits the performance of modern plasmonic devices.

Localized surface plasmon resonances occur in plasmonic nanoparticles with sizes smaller than, or comparable to, the wavelength of light. When the plasmonic nanoparticle is excited with light at resonance wavelength, collective oscillation of electrons provide large field enhancement and high local temperatures near the particle. Therapeutic applications which are based on the destruction of unwanted cells make use of local high temperatures provided by plasmonic nanostructures that are excited by a directed light source.

The amount of heat power delivered to the local medium is directly proportional to the power of illuminating light where the proportionality constant is the absorption cross-section of the plasmonic nanoparticle (Baffou, G. and R. Quidant, “Thermo-plasmonics: using metallic nanostructures as nano-sources of heat,” Laser & Photonics Reviews, 2013. 7(2): pp. 171-1871. The absorption cross-section of a nanoparticle is the amount of electromagnetic energy dissipated into heat inside the particle.

Conventionally, gold and silver have been the metals of choice for plasmonic devices, due to their lower optical losses. However, gold and silver are still not the best materials to fabricate and integrate into plasmonic devices because of several other problems associated with the metals. First, their optical losses are small but certainly not insignificant. In visible range, the losses are relatively high for gold due to interband absorption. Additionally, gold and silver do not have optical properties that may be tuned to suit a particular application. Second, gold and silver are difficult to fabricate into ultra-thin films, which are often necessary in plasmonic devices. Third, silver and gold are not thermally stable at high temperatures, especially when nanostructured. Fourth, silver is not chemically stable and causes problems in many applications such as sensing (Guler, U. and R. Turan, Effect of particle properties and light polarization on the plasmonic resonances in metallic nanoparticles. Opt Express, 2010. 18(16): p. 17322-38). Fifth, neither metal is CMOS compatible, hence posing challenges in the integration of plasmonic devices with nanoelectronic CMOS devices.

The problems associated with gold and silver severely limit the development of plasmonics as a science into a technology. Hence, alternative plasmonic materials are essential to the further development of this technology.

### SUMMARY OF THE INVENTION

In one embodiment, a nanoparticle material is made of a one or more particles comprising a core material covered with a shell layer. The core is titanium nitride (TiN) and the shell layer is made of TiO₂, the TiN providing localized surface plasmon resonances (LSPR) in a biological transparency window. The outer TiO₂ layer provides both (1) a buffer layer for surfactant coupling; and (2) a mechanism for shifting a resonance of the nanoparticle, thus allowing for resonance control (or adjustment, tuning, change, etc.). In other embodiments, the material comprises a TiO₂ core and a TiN shell layer for further improved resonance control.

The material making up each nanoparticle is capable of synthesis at temperatures above 300 degrees Celsius.

In some embodiments, the TiO₂ shell layer is produced by an oxidation of the TiN. In other embodiments, the nanoparticle further comprises one or more surfactants coupled to the external surface of the shell layer in order to bind specific target sites.

One or more surfactants may have a shape which provides its attachment to a defective cell in a human body. The surfactant(s) may also provide drug delivery to a specific place in a human body. The particles may be simple geometric shapes (e.g., cube, spehere, etc.).

The size of a particle may be less than, about, or greater than 1 nm.

The material may be, fabricated using lithographic methods and creating an array of nanoparticles fixed on a substrate (e.g. a chip).

The material is not limited to titanium nitride or titanium oxide, and may further comprise transition metal nitrides, oxides, carbides, borides, sulfides, halides, or a combination thereof.

A method destroying a defective cell in a human body, employing local-heating clinical therapeutic application, is also disclosed herein. The method comprises chemically synthesizing titanium nitride nanoparticles (101), coupling surfactants to the nanoparticles (102), injecting said nanoparticles with coupled surfactants into a body having the defective cell (103), wherein the surfactants help bind said nanoparticles to the defective cell. Then, by directing an electromagnetic radiation at said nanoparticles from an external source of radiation, emitted at a resonant wavelength corresponding to a resonance of said nanoparticles, energy is delivered to the nanoparticles, thus raising a temperature of said nanoparticles to form a heat source (104). This heat source increases a temperature of the defective cell to destroy only the defective cell without affecting a surrounding tissue (105).

In some embodiments, the method destroys a cancer cell. In other embodiments, the method destroys fat cell. In yet other embodiments, the method employs particles which remain stable after multiple electromagnetically induced heatings to a temperature of 50 degrees Celsius or higher. The particles may comprise chemically synthesized titanium nitride nanoparticles surrounded by chemically synthesized TiO₂ shell layers, or vice versa. Additionally, more surfactants may be coupled to said nanoparticles, the additional surfactants delivering a drug to the defective cells. The nanoparticles may further act as nanometer scale optical antennas for bin-imaging and bio-sensing.

### BRIEF DESCRIPTION OF DRAWINGS

FIG. 1 shows examples of some of the possible nanoparticle geometries that may be fabricated by the above-mentioned methods and used for biological testing and treatment. Specifically, FIG. 1 shows a plasmonic TiN (a) nanoparticle, (b) nanoshell, (c) inverted nanoshell. FIG. 1(d) shows a TiN particle surface modified with surfactants. FIG. 1(e) shows a TiN nanoparticle array fabricated with lithographic methods on top of a substrate. FIG. 1(f) shows a cube nanoparticle with coupled surfactants.

FIG. 2. Transmittance of TiN nanoparticle arrays grown at (a) 400 degrees Celsius, and (b) 800 degrees Celsius, showing extinction dips due to plasmonic resonances in the biological window of the electromagnetic spectrum. The solid black lines show the edges of the generally accepted biological transparency window.

FIG. 3: Absorption efficiencies of TiN nanoparticles grown at (a) 400 degrees Celsius, (b) 800 degrees Celsius, and (c) absorption efficiencies of Au nanoparticles. The dashed line shows the 800 nm wavelength, which is in the biological transparency window and is widely used in therapeutic applications. (d) Time dependent temperature measurement of Sapphire substrate patterned with identical nanoparticle arrays of Au and TiN, grown at 400 degrees Celsius and 800 degrees Celsius.

FIG. 4: (a) An illustration of a cancer cell covered by one embodiment of the nanoparticles disclosed herein, and illuminated with a light beam at near-resonance wavelengths in the biological transparency window. (b) An illustration of a cancer cell damaged by the heat generated by one embodiment of the nanoparticles disclosed herein.

FIG. 5: (a) A high resolution TEM image of a nanoparticle after the nitridation process. (b) A diffraction pattern taken from the region marked by a dashed rectangle in FIG. 5(a). (c) A calculated diffraction pattern from tabulated data. (d) An optical transmittance spectrum of nanoparticles before and after the nitridation process. The broad plasmonic peak covers visible and near infrared regions.

FIG. 6: (a) An optical transmittance spectrum of TiN nanoparticles before and after the annealing process. Both examples exhibit plasmonic resonances, and the annealing process may be used for modification of optical properties. (b) A high resolution TEM image of one embodiment of a TiN nanoparticle. (c) A diffraction pattern obtained from the individual nanoparticle shown in FIG. 6(b). (d) A simulated diffraction pattern from tabulated data for one type of crystalline TiN.

### DETAILED DESCRIPTION OF THE PREFERRED EMBODIMENT

This invention provides a new approach to nanoparticle-based plasmonic solutions to therapeutic applications by use of titanium nitride (TiN) as the plasmonic material. Employment of TiN nanoparticles in such applications enables usage of particles with simple geometries and small sizes. In addition, the broad resonance characteristics of TiN nanoparticles eliminate the size dispersion restrictions. In current applications where gold (Au) is employed as the plasmonic material, complex shapes and large particle sizes are considered in order to get plasmonic resonances in the biological window. Also, relatively narrower plasmonic peaks with Au create the requirement of having nanoparticles with a very narrow size dispersion. TiN nanoparticles provide plasmonic resonances occurring in the biological window even with small sizes. Local heating efficiencies of TiN nanoparticles outperform currently used Au nanoparticles. The use of smaller particles with simpler shapes and better heating efficiencies allows better diffusion properties into tumor regions, larger penetration depth of light into the biological tissue, and the ability to use excitation light with less power.

One of the alternatives that resemble the optical properties of gold is titanium nitride (TiN). Titanium nitride is one of the hardest materials with a very high melting point (>2700° C.). TiN is CMOS compatible, bio-compatible, and may be grown as high quality ultra-thin films or as nanostructured films. These advantages of TiN make it a better alternative plasmonic material. TiN was demonstrated to support surface plasmon-polaritons (SPPs), and TiN nanostructures exhibit localized surface plasmon resonance (LSPR) (Naik, G. V., et al., Titanium nitride as a plasmonic material for visible and near-infrared wavelengths. Optical Materials Express, 2012. 2(4): p. 478-489).

The strength of the LSPR in TiN nanoparticles is similar to that of gold nanoparticles, but occurring in a broad wavelength range around 850 nm (Guler, U., et al., Performance analysis of nitride alternative plasmonic materials for localized surface plasmon applications. Applied Physics B, 2012. 107(2): p. 285-291). This corresponds to the biological transparency window which most bio- and medical applications cover. Often, bio-and medical applications involving plasmons utilize LSPR in metal nanoparticles. LSPR enhances the electromagnetic field around the nanoparticle by many times, and it also causes the metal particle to absorb much more radiation than it would without LSPR. Such excessive absorption of optical radiation causes the nanoparticle to locally heat its surroundings. Local heating is useful in applications such as selective killing of unwanted cells including, but not limited to, cancer cells, fat cells, etc., as well as more efficient heating for energy harvesting including, but not limited to, solar steam generation, thermophotovoltaics, etc. TiN nanoparticles are a better substitute to gold nanoparticles given their bio-compatibility, thermal stability, comparable heating performance, and LSPR occurring in the biological transparency window. Both experimental and numerical results show that TiN performs better than gold in the biological window for heating applications.

TiN nanoparticles may be produced using several different methods, including both top-down and bottom-up approaches. Studies on lithographically fabricated TiN nanoparticles show superior plasmonic characteristics when compared to Au in the biological window of the electromagnetic spectrum. It has also been shown that TiN powders may be obtained by means of chemical synthesis with several different methods including both high and low temperature processes (D'Anna, E., et al., Oxidation interference in direct laser nitridation of titanium: relative merits of various ambient gases. Thin Solid Films 1992. 213(2): p. 197-204; Giordano, C., et al., Metal Nitride and Metal Carbide Nanoparticles by a Soft Urea Pathway. Chemistry of Materials, 2009. 21(21): p. 5136-5144; Gomathi, A. and C. N. R. Rao, Nanostructures of the binary nitrides, BN, TiN, and NbN, prepared by the urea-route. Materials Research Bulletin, 2006. 41(5): p. 941-947.; Hu, J., et al., Low-Temperature Synthesis of Nanocrystalline Titanium Nitride via a Benzene-Thermal Route. Journal of the American Ceramic Society, 2000. 83(2): p. 430-432; Li, J., et al., Synthesis of Nanocrystalline Titanium Nitride Powders by Direct Nitridation of Titanium Oxide. Journal of the American Ceramic Society, 2001. 84(12): p. 3045-3047; Yana, X., et al., Reduction-Nitridation Synthesis of Titanium Nitride Nanocrystals. Journal of the American Ceramic Society, 2003. 86(1): p. 206-208).

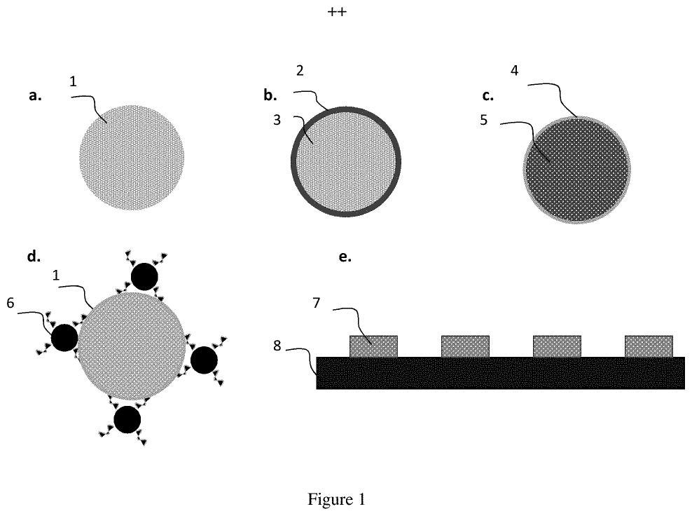

FIG. 1 shows some of the possible nanoparticle geometries that may be fabricated by the above-mentioned methods and used for biological testing and treatment.

FIG. 1.a shows the simplest embodiment of the present invention, which is a spherical TiN nanoparticle (1) that may have a wide range of dimensions starting from a few nanometers. Such nanoparticles are chemically synthesized and, although illustrated here as a sphere, may be any shape. Simple shapes and small particle sizes are preferred in biological applications where diffusive properties of these particles are important. The spectral position of TiN plasmonic resonance allows for the use of simpler shapes with smaller sizes.

FIG. 1.b and FIG. 1.c show the nanoshell arrangement widely used with Au nanoparticles due to the mismatch of the plasmonic resonance of Au and the transparency window for biological samples. Even though the simple case of a spherical particle satisfies the resonance conditions with TiN, it may be possible to fabricate nanoshells with a TiN core (3) and a TiO₂ shell layer (2), as well as inverted configurations, such as a TiO₂ core (5) with a TiN shell layer (4), where needed. The TiO₂ shell layer (2) may be obtained by oxidizing a TiN particle, or by other chemical methods. The TiN shell layer may be obtained by nitridizing a TiO₂ particle, or by other chemical methods. The TiO₂ shell (or any shell material) has a double function. The TiO₂ provides a buffer layer (FIG. 1.b, (2)) to which one or more surfactants (6) may be coupled or linked (“surfactant coupling”). The TiO₂ also simultaneously provides a mechanism for shifting the resonance of a nanoparticle, thus acting as a resonance control means for the nanoparticle. The nanoparticle size may be less than, about, or greater than 1 nanometer in length along any cross-section or distance through the nanoparticle.

In addition to TiN and TiO₂, the nanoparticle may further alternatively comprise other materials including but not limited to transition metal nitrides, oxides, carbides, borides, sulfides, halides, or a combination thereof.

FIG. 1.d shows how the surface of the TiN nanoparticle may be modified with surfactants (6) in order to deliver these particles to the tumor region. These surfactants are molecules used for surface modification of the actual nanoparticle to benefit the delivery process. Any known method of delivery may be used to transport the nanoparticles to a tumor region (See, e.g., Johnston, K.P.D.T.A.T.X., et al., MEDICAL AND IMAGING NANOCLUSTERS, T.H.E.U.O.F.T.S.W.t.S.A.T.X. Board Of Regents, Editor, 2010: WO; Vitaliano, F.B.M.A., BIO-NANO-PLASMONIC ELEMENTS AND PLATFORMS. 2012: US; Bhatia, S.N.I.R.L.M.A., et al., DELIVERY OF NANOPARTICLES AND/OR AGENTS TO CELLS, M.A.C.M.A. Massachusetts Institute Of Technology, S.A.N.D.T.T. University Of California, and M.C.L.J.C.A. Ip Services Gilman Drive, Editors. 2008: WO). Furthermore, the surfactants may be of any shape, whether it is a regular shape (6) or an irregular shape (20). The shape of the surfactant correlated to the intended function of the nanoparticle. For example, some surfactants may be specifically shaped to enable attachment to a specific type of defective cell in a human body. The shape may be a simple geometric shape (e.g., cube, sphere, etc.) or an irregular shape (any random formation, specifically engineered with an affinity to certain places or cells within a human or other target body). The surfactant may alternatively be engineered (based on shape or another parameter) in order to deliver a drug to a specific area/location within the human body (rather than heating, or in addition to heating, as described herein). By injecting (or otherwise administering) the nanoparticles with surfactants into a human or other target body containing a defective target cell, the nanoparticle is able to bind the defective target cell via the surfactants designed specifically for that nanoparticle's function. Once the nanoparticle is in proximity to the defective target cell, electromagnetic radiation may be emitted towards the nanoparticle(s) within the body (at the resonant wavelength corresponding to the nanoparticle's material), causing the nanoparticle(s) to increase in temperature and creating a heat source within the body. The heat source near the defective cell in turn causes a temperature rise within the defective cell, thus causing the cell to overheat and destroying the cell. It should be noted, as well, that the nanoparticle does not reach a temperature or a proximity to any healthy or other surrounding tissue not targeted by the process. Thus, only the defective target cell destroyed, while all other surrounding tissue remains unharmed and unaffected. The defective cell may be, for example, a cancer cell(s) or a fat cell(s). Multiple (i.e. more than 5, 10, 15, 20, 25, 30. etc.) individual heatings, at 50 degrees Celsius or higher, may be withstood by the nanoparticle material disclosed herein, without the nanoparticle losing its stability, shape, and function.

FIG. 1.e illustrates TiN nanoparticles (7), which may be of any shape and size, and that may be fabricated by lithographic methods. Lithographically fabricated particles help in examining the properties of plasmonic structures due to their high precision capabilities. These two-dimensional array structures may also be employed in on-chip applications for biological testing (i.e. TiN nanoparticle array (7), fabricated using lithographic methods, grown on top of a substrate (8)). The substrate (8) may be made of, e.g., glass, sapphire, MgO, etc. FIG. 1.f illustrates a nanoparticle in the shape of a cube (21), coupled to surfactants (6) on its external surface. Any number of surfactants may be coupled to the particle.

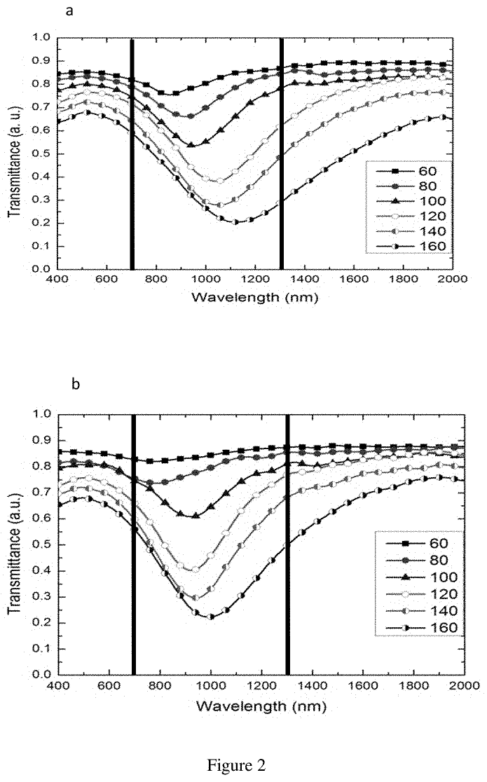

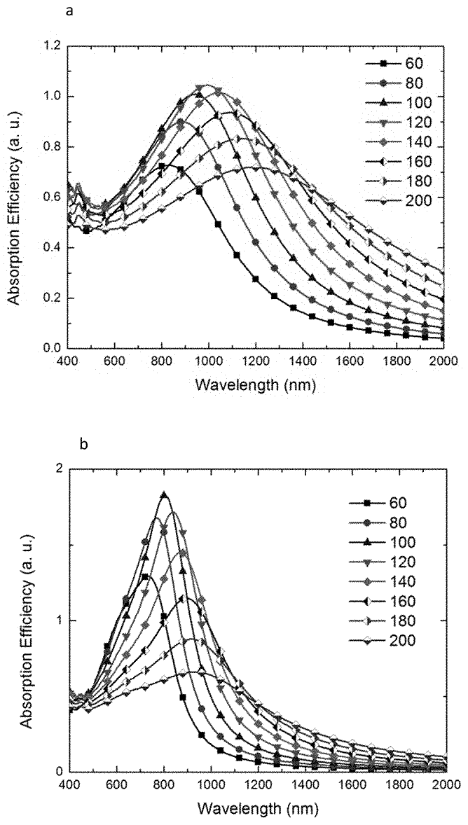

FIG. 2 shows the transmittance curve of TiN nanoparticle arrays fabricated with electron beam lithography, which allows for particles with precise dimensions and well-defined geometries. TiN nanoparticles grown at (a) 400° C. and (b) 800° C. have plasmonic resonances covering the biological transparency window, as may be observed from the transmittance curves provided in this figure. The vertical lines show the range of the generally accepted biological transparency window. The extinction dips are due to plasmonic resonances in the biological window of the electromagnetic spectrum. It should be noted that the core and shell materials may be synthesized at any temperature at or above about 300 degrees Celsius. The specific temperature examples provided herein do not limit the present disclosure.

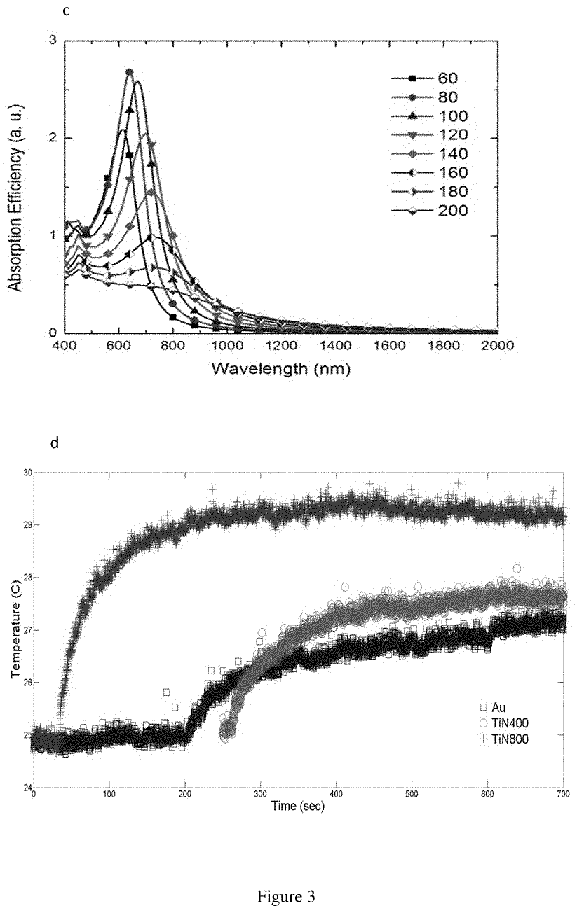

FIGS. 3.a-3.c show the numerical results for the absorption efficiencies of TiN and Au nanoparticles. FIG. 3.a shows results for TiN grown at 400° C., FIG. 3.b shows results for TiN grown at 800° C. and FIG. 3.c shows results for Au nanoparticles. TiN nanoparticles provide better efficiencies in the biological transparency window. The non-stoichiometric nature of TiN enables the tunability of plasmonic properties with growth conditions. Both FIGS. 3.a-3.c and FIG. 2 illustrate that the resonance of the nanoparticles according to the present invention may be tuned to specific wavelengths in order to perform resonance control when and if necessary (to match the resonance of the targeting emitted electromagnetic radiation).

FIG. 3.d shows a time-dependent temperature measurement of a sapphire substrate patterned with nanoparticles of identical shapes (nanoparticles made of either Au, TiN grown at 400° C., or TiN grown at 800° C.) when illuminated with a laser beam at an 800 nm wavelength. The results show that TiN performs better than Au for heating applications and agree with the simulations that optical properties may be tuned by adjusting the growth temperature.

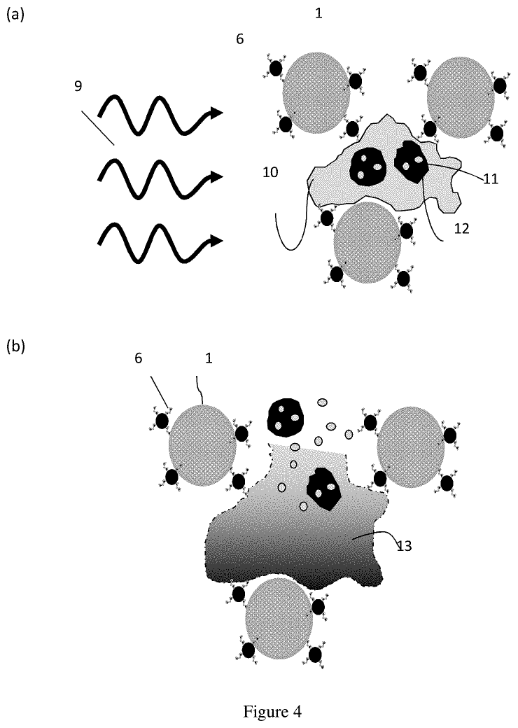

FIG. 4 illustrates an example of TiN nanoparticles (1) injected into a target body (22) (human or other), modified with surfactants (6) which help attach the nanoparticles (1) to a cancer cell (10) (or any other defective or target cell), containing a nucleus (11) and nucleolus (12). After the delivery of TiN nanoparticles (1) to the tumor region (10), a beam of light (9), with visible/NIR (near-infrared range) wavelengths, such as a laser beam, at the resonance wavelength of these nanoparticles, is directed towards the same region in order to deliver energy to the nanoparticles (the radiation may occur once or any number of times, depending on the desired result and application). Since the plasmonic resonance of these nanoparticles is within the biological transparency window, incident light reaches deeper regions within the specimen and is absorbed by the TiN nanoparticles. The electromagnetic energy absorbed by the particles is transferred into heat and creates a heat source within the nanoparticle (1) and thus increases the local temperature. Cancer or other defective/target cells (10), located very close to TiN nanoparticles, thus begin to experience elevated temperatures. Using plasmonic nanoparticles, it is possible to destroy target cells attached to and/or near the nanoparticles by, e.g., breaking the cell membrane (as shown in FIG. 4.b, (23)), without damaging the sample and any surrounding healthy tissue or cells (13). Only a destroyed defective cell (10) exists after photo thermal treatment.

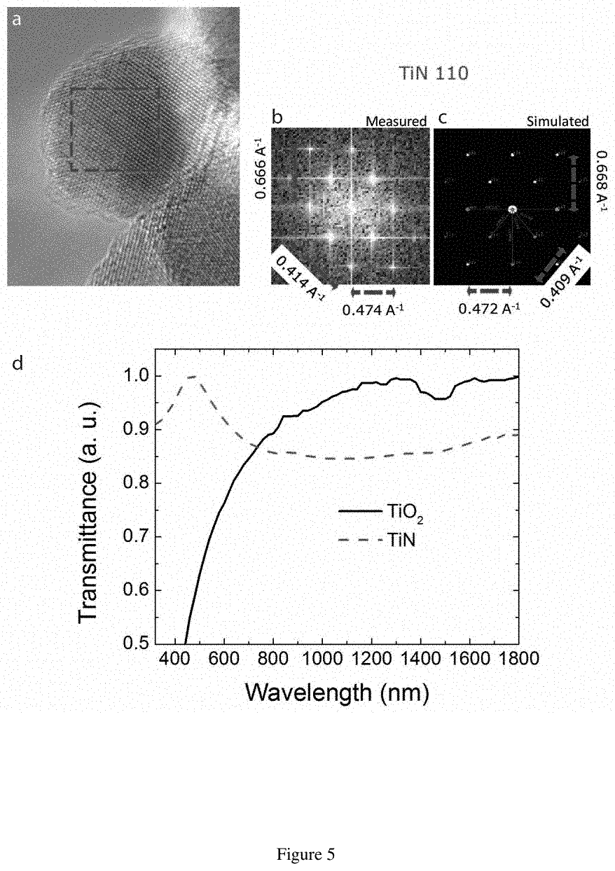

In the preferred embodiment the nanoparticles are obtained by the following method. Direct nitridation of TiO₂ nanoparticles at temperatures above 700° C. in a nitrogen rich environment such as NH₃ is used as an efficient method for obtaining TiN nanoparticles with plasmonic properties that lead to feasible nanoparticle thermal therapy. Process duration varies between 1 and 15 hours depending on the desired batch size and properties as well as other process parameters. TiN nanoparticles with a cubic crystalline structure are obtained via nitridaton of TiO₂ nanoparticles, see FIGS. 5(a), 5(b), and 5(c), and extinction dips due to plasmonic resonance in the visible and near infrared region are observed in the optical transmittance data, see FIG. 5(d). The range in size of the product nanoparticles depends on the starting TiO₂ nanoparticle dimensions and process parameters such as duration, temperature, and gas flow rate.

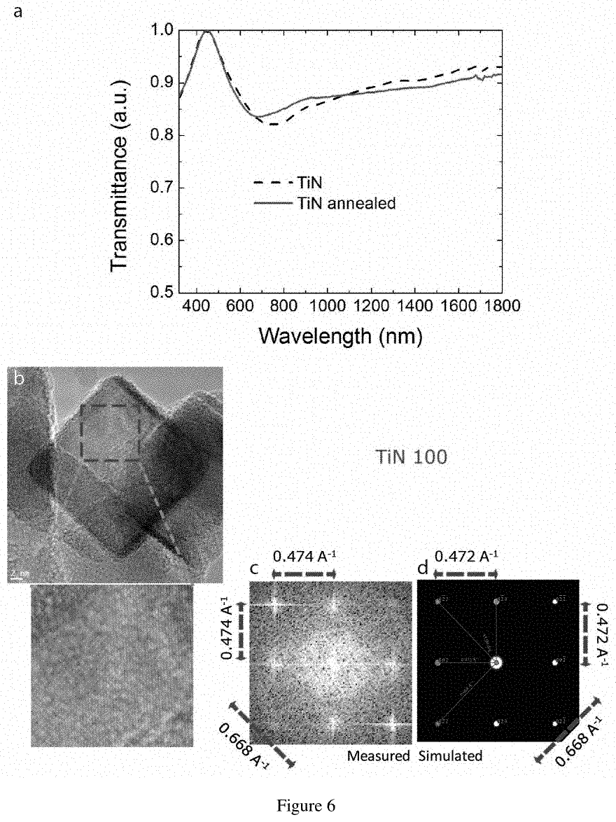

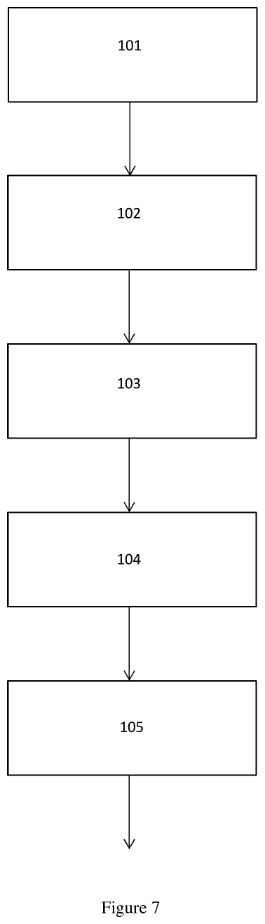

In another embodiment, another method of producing plasmonic TiN nanoparticles is used: a plasma arc method, where Ti nanostructures are processed in a nitrogen rich environment. FIG. 6(a) shows the plasmonic extinction dips obtained from samples produced using a plasma arc method. The optical properties may be further modified by a direct nitridation method. FIGS. 6(b), 6(c) and 6(d) show an example of the cubic crystalline structure of such nanoparticle samples.

It should additionally be noted, that the nanoparticles (or nanoparticle material) described herein may additionally be used as nanometer scale optical antenna for bio-imaging and bio-sensing applications. By similar injection into a human body, the nanoparticles' resonance or other properties may be monitored in order to obtain information of nanometer scale processes and components within a human or other target body.

The description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

Moreover, the words “example” or “exemplary” at used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

1. A method of destroying a defective cell in a human body, for local-heating clinical therapeutic application, comprising:
chemically synthesizing titanium nitride nanoparticles;
coupling surfactants to said nanoparticles;
injecting said nanoparticles with coupled surfactants into a body having the defective cells;
said surfactants binding said nanoparticles to the defective cell;
directing an electromagnetic radiation at said nanoparticles from an external source of radiation, wherein said radiation is emitted at a resonant wavelength corresponding to a resonance of said nanoparticles resonance, thus delivering energy to the nanoparticles and raising a temperature of said nanoparticles to form a heat source;
wherein said heat source increases a temperature of the defective cell to destroy only the defective cell without seriously affecting a surrounding tissue.

2. The method of claim 1, wherein the defective cell is a cancer cell.

3. The method of claim 1, wherein the defective cell is a fat cell.

4. The method of claim 1, wherein the nanoparticles remain stable after multiple electromagnetically induced heatings to a temperature of 50 degrees Celsius or higher.

5. The method of claim 4, wherein the nanoparticles are chemically synthesized TiN nanoparticles further comprising a chemically synthesized TiO2 shell layer surrounding each said TiN nanoparticle.

6. The method of claim 1, further comprising coupling additional surfactants to said nanoparticles, the additional surfactants delivering a drug to the defective cell.
